SEQUENCE LISTING (68 sequences)

SEQ ID NO: 1 - protein, 37 aa, Xenopus laevis
  VARIANT (37)..(37): Optional amidation
HAEGTFTSDVTQQLDEKAAKEFIDWLINGGPSKEIIS

SEQ ID NO: 2 - protein, 37 aa, Xenopus laevis
  VARIANT (37)..(37): Optional amidaton
HAEGTYTNDVTEYLEEKAAKEFIEWLIKGKPKKIRYS

SEQ ID NO: 3 - protein, 30 aa, Homo sapiens
  VARIANT (30)..(30): Optional amidation
HAEGTFTSDVSSYLEGQAAKEFIAWLVKGR

SEQ ID NO: 4 - protein, 39 aa, Heloderma suspectum
  VARIANT (39)..(39): Optional amidation
HGEGTFTSDLSKQMEEEAVRLFIEWLKNGGPSSGAPPPS

SEQ ID NO: 5 - protein, 28 aa, Heloderma suspectum
  VARIANT (28)..(28): Optional amidation
HGEGTFTSDLSKQMEEEAVRLFIEWLKN

SEQ ID NO: 6 - protein, 31 aa, Artificial Sequence
  Synthetic peptide
HAEGTFTNDMTNYLEEKAAKEFVGWLIKGRS

SEQ ID NO: 7 - protein, 28 aa, Artificial Sequence
  Synthetic peptide
HAEGTFTSDVTQQLDEKAAKEFIDWLIN

SEQ ID NO: 8 - protein, 28 aa, Artificial Sequence
  Synthetic peptide
HXEGTFTSDVTQQLDEKAAKEFIDWLIN

SEQ ID NO: 9 - protein, 39 aa, Artificial Sequence
  Synthetic peptide
HAEGTFTSDVTQQLDEKAAKEFIDWLINGGPSSGAPPPS

SEQ ID NO: 10 - protein, 37 aa, Artificial Sequence
  Synthetic peptide
HXEGTFTSDVTQQLDEKAAKEFIDWLINGGPSKEIIS

SEQ ID NO: 11 - protein, 37 aa, Artificial Sequence; identical to SEQ ID NO: 1
  Synthetic peptide

SEQ ID NO: 12 - protein, 37 aa, Artificial Sequence
  Synthetic peptide
HAEGTFTSDVTQQLDEKAAKEFIDWLINGGASKEIIS

SEQ ID NO: 13 - protein, 37 aa, Artificial Sequence
  Synthetic peptide
HAEGTFTSDVTQQLDEKAAKEFIDALINGGPSKEIIS

SEQ ID NO: 14 - protein, 37 aa, Artificial Sequence
  Synthetic peptide
HAEGTFTSDVTQQLDEKAAKEFIDWLINPGSEGIKSI

SEQ ID NO: 15 - protein, 37 aa, Artificial Sequence
  Synthetic peptide
HXEGTFTSDVTQQLDEKAAKEFIDWLINGGPSKEIIX

SEQ ID NO: 16 - protein, 37 aa, Artificial Sequence
  Synthetic peptide
HXEGTFTSDVTQQLDEKAAXEFIDWLINGGPSKEIIS

SEQ ID NO: 17 - protein, 37 aa, Artificial Sequence; identical to SEQ ID NO: 16
  Synthetic peptide

SEQ ID NO: 18 - protein, 37 aa, Artificial Sequence; identical to SEQ ID NO: 16
  Synthetic peptide

SEQ ID NO: 19 - protein, 39 aa, Artificial Sequence
  Synthetic peptide
HAEGTYTNDVTEYLEEKAAKEFIEWLIKGGPSSGAPPPS

SEQ ID NO: 20 - protein, 39 aa, Artificial Sequence
  Synthetic peptide
HXEGTYTNDVTEYLEEKAAKEFIEWLIKGGPSSGAPPPS

SEQ ID NO: 21 - protein, 37 aa, Artificial Sequence
  Synthetic peptide
HAEGTYTNDVTEYLEEKAAKEFIEWLINGGPSKEIIS

SEQ ID NO: 22 - protein, 37 aa, Artificial Sequence
  Synthetic peptide
HAEGTFTSDVTQQLEEKAAKEFIEWLIKGKPKKIRYS

SEQ ID NO: 23 - protein, 37 aa, Artificial Sequence
  Synthetic peptide
HXEGTYTNDVTEYLEEKAAKEFIEWLIKGKPKKIRYS

SEQ ID NO: 24 - protein, 28 aa, Artificial Sequence
  Synthetic peptide
HAEGTYTNDVTEYLEEKAAKEFIEWLIN

SEQ ID NO: 25 - protein, 28 aa, Artificial Sequence
  Synthetic peptide
HAEGTYTNDVTEYLEEKAAKEFIEWLIK

SEQ ID NO: 26 - protein, 37 aa, Artificial Sequence
  Synthetic peptide
HGEGTYTNDVTEYLEEKAAKEFIEWLIKGKPKKIRYS

SEQ ID NO: 27 - protein, 37 aa, Artificial Sequence; identical to SEQ ID NO: 23
  Synthetic peptide

SEQ ID NO: 28 - protein, 37 aa, Artificial Sequence; identical to SEQ ID NO: 23
  Synthetic peptide

SEQ ID NO: 29 - protein, 36 aa, Artificial Sequence
  Synthetic peptide
HAEGTYTNDVTEYLEKAAKEFIEWLIKGKPKKIRYS

SEQ ID NO: 30 - protein, 36 aa, Artificial Sequence
  Synthetic peptide
HAEGTYTNDVTEYLEEAAKEFIEWLIKGKPKKIRYS

SEQ ID NO: 31 - protein, 36 aa, Artificial Sequence
  Synthetic peptide
HAEGTYTNDVTEYLEEKAAKFIEWLIKGKPKKIRYS

SEQ ID NO: 32 - protein, 34 aa, Artificial Sequence
  Synthetic peptide
HAEGTYTNDVTEYLEEKAAKEFIEWLIPKKIRYS

SEQ ID NO: 33 - protein, 36 aa, Artificial Sequence
  Synthetic peptide
HAEGTYTNDVTELEEKAAKEFIEWLIKGKPKKIRYS

SEQ ID NO: 34 - protein, 36 aa, Artificial Sequence
  Synthetic peptide
HAEGTYTNDVTEYLEEKAAKEFIEWLIKGKPKKIRY

SEQ ID NO: 35 - protein, 36 aa, Artificial Sequence
  Synthetic peptide
HAEGTYTNDVTEYLEEKAAKEFIEWLIGKPKKIRYS

SEQ ID NO: 36 - protein, 36 aa, Artificial Sequence
  Synthetic peptide
HAEGTYTNDVTEYLEEKAAKEFIEWLIKGPKKIRYS

SEQ ID NO: 37 - protein, 35 aa, Artificial Sequence
  Synthetic peptide
EGTYTNDVTEYLEEKAAKEFIEWLIKGKPKKIRYS

SEQ ID NO: 38 - protein, 36 aa, Artificial Sequence
  Synthetic peptide
HAEGTYTNDVTEYEEKAAKEFIEWLIKGKPKKIRYS

SEQ ID NO: 39 - protein, 36 aa, Artificial Sequence
  Synthetic peptide
HAEGTYTNDTEYLEEKAAKEFIEWLIKGKPKKIRYS

SEQ ID NO: 40 - protein, 32 aa, Artificial Sequence
  Synthetic peptide
HAEGTYTNDVTEYLEEKAAKEFIEWLIKGKPK

SEQ ID NO: 41 - protein, 36 aa, Artificial Sequence
  Synthetic peptide
HAEGTYTNDVTYLEEKAAKEFIEWLIKGKPKKIRYS

SEQ ID NO: 42 - protein, 27 aa, Artificial Sequence
  Synthetic peptide
HAEGTYTNDVTEYLEEKAAKEFIEWLI

SEQ ID NO: 43 - protein, 27 aa, Artificial Sequence
  Synthetic peptide
HXEGTYTNDVTEYLEEKAAKEFIEWLI

SEQ ID NO: 44 - protein, 26 aa, Artificial Sequence
  Synthetic peptide
HAEGTYTNDVTEYLEEKAAKEFIEWL

SEQ ID NO: 45 - protein, 26 aa, Artificial Sequence
  Synthetic peptide
HXEGTYTNDVTEYLEEKAAKEFIEWL

SEQ ID NO: 46 - protein, 25 aa, Artificial Sequence
  Synthetic peptide
HAEGTYTNDVTEYLEEKAAKEFIEW

SEQ ID NO: 47 - protein, 25 aa, Artificial Sequence
  Synthetic peptide
HXEGTYTNDVTEYLEEKAAKEFIEW

SEQ ID NO: 48 - protein, 38 aa, Ictalurus punctatus
  VARIANT (38)..(38): Optional amidation
HADGTYTSDVSSYLQDQAAKDFITWLKSGQPKPDAVET

SEQ ID NO: 49 - protein, 44 aa, Ictalurus punctatus
  VARIANT (44)..(44): Optional amidation
HADGTYTSDVSSYLQDQAAKDFITWLKSGQPKPDAVETRSVDTL

SEQ ID NO: 50 - protein, 37 aa, Hoplobatrachus rugulosus
  VARIANT (37)..(37): Optional amidation
HAEGTYTNDVTQFLEEKAAKEFIDWLIKGKPKKQRLS

SEQ ID NO: 51 - protein, 28 aa, Amia calva
  VARIANT (28)..(28): Optional amidation
YADAPYISDVYSYLQDQVAKKWLKSGQD

SEQ ID NO: 52 - protein, 32 aa, Petromyzon marinus
  VARIANT (32)..(32): Optional amidation
HADGTFTNDMTSYLDAKAARDFVSWLARSDKS

SEQ ID NO: 53 - protein, 37 aa, Artificial Sequence
  Synthetic peptide
HXEGTYTNDVTQFLEEKAAKEFIDWLIKGKPKKQRLS

SEQ ID NO: 54 - protein, 37 aa, Artificial Sequence; identical to SEQ ID NO: 53
  Synthetic peptide

SEQ ID NO: 55 - protein, 37 aa, Artificial Sequence
  Synthetic peptide
HGEGTYTNDVTQFLEEKAAKEFIDWLIKGKPKKQRLS

SEQ ID NO: 56 - protein, 37 aa, Artificial Sequence; identical to SEQ ID NO: 53
  Synthetic peptide

SEQ ID NO: 57 - protein, 9 aa, Artificial Sequence
  Synthetic peptide
GGPSKEIIS

SEQ ID NO: 58 - protein, 9 aa, Artificial Sequence
  Synthetic peptide
GKPKKIRYS

SEQ ID NO: 59 - protein, 11 aa, Artificial Sequence
  Synthetic peptide
GGPSSGAPPPS

SEQ ID NO: 60 - protein, 9 aa, Artificial Sequence
  Synthetic peptide
PGSEGIKSI

SEQ ID NO: 61 - protein, 10 aa, Artificial Sequence
  Synthetic peptide
GQPKPDAVET

SEQ ID NO: 62 - protein, 8 aa, Artificial Sequence
  Synthetic peptide
IDWLIKGQ

SEQ ID NO: 63 - protein, 9 aa, Artificial Sequence
  Synthetic peptide
IDWLIKGQG

SEQ ID NO: 64 - protein, 16 aa, Artificial Sequence
  Synthetic peptide
GQPKPDAVETRSVDTL

SEQ ID NO: 65 - protein, 9 aa, Artificial Sequence
  Synthetic peptide
GKPKKQRLS

SEQ ID NO: 66 - protein, 8 aa, Artificial Sequence
  Synthetic peptide
YADAPYIS

SEQ ID NO: 67 - protein, 15 aa, Artificial Sequence
  Synthetic peptide
VGGGSTMEDDTEPLS

SEQ ID NO: 68 - protein, 15 aa, Artificial Sequence
  Synthetic peptide
GQPKQDVGTNRGTNL

(19) United States
(12) Patent Application Publication
(10) Pub. No.: US 2010/0009907 A1
(43) Pub. Date: Jan. 14, 2010

(54) Glucagon-Like Peptides and Uses Thereof

(75) Inventors: Richard A. Pittner, San Diego, CA (US); Ved Srivastava, San Diego, CA (US); Samuel Janssen, San Diego, CA (US)

Correspondence Address: Intellectual Property Department;Amylin Pharmaceuticals, Inc., 9360 Towne Centre Drive, San Diego, CA, 92121, US

(73) Assignee: AMYLIN PHARMACEUTICALS, INC., San Diego, CA (US)

(21) Appl. No.: 12/302,456

(22) PCT Filed: Jul. 6, 2007
(86) PCT No.: PCT/US07/15565

§ 371 Date: Apr. 15, 2009

Related U.S. Application Data
(60) Provisional application No. 60/818,732, filed on Jul. 6, 2006.

(51) Int. Cl. A61P 7/12; C07K 7/06; C07K 7/08; C07K 14/00; A61P 3/00; C07K 14/76; C07K 16/00; C07H 21/04; A61K 38/08; A61K 38/10; A61K 38/16; A61P 1/00; A61P 11/00

(52) U.S. Cl. 514/12; 530/328; 530/326; 530/327; 530/324; 530/362; 530/387.3; 536/23.1; 514/16; 514/15; 514/14; 514/13

(57) ABSTRACT

The present invention relates to non-mammalian Glucagon-like peptides (nmGLP-1), analogs thereof, related nucleic acids, and processes production of such peptides. The nmGLP-1 peptides and analogs thereof of the invention include one or more amino acid sequence modifications. In addition, methods and compositions are disclosed to treat and prevent obesity, reducing body weight, controlling glycemia, controlling or reducing appetite, and reducing food intake.

### FIELD OF THE INVENTION

The present application relates to non-mammalian glucagon-like peptides, analogs, and agonist analogs thereof including uses, compositions, and methods of administration thereof.

### BACKGROUND OF THE INVENTION

Several peptides normally secreted from the gastrointestinal tract during eating have been shown to suppress food intake if given before meals. During recent years, the role of the pre-absorptive release of gut peptides (especially cholecystokinin, bombesin-like peptides and glucagon-like peptides) in the production of meal-ending satiety has been extensively investigated in animals.

Human Glucagon-like peptide-1 (7-36) amide (GLP-1), the biologically active form of the GLP-1 protein resulting from a post-translational modification of the pro-hormone pro-glucagon, is released from enteroendocrine cells from the distal gut in response to food intake. While human GLP-1 has been shown to be effective in appetite control in human beings, there is a need to develop and administer variants of human GLP-1 protein. There is also a need to find additional uses for variants of human GLP-1 protein.

### SUMMARY OF THE INVENTION

The present invention relates, at least in part, to novel non-mammalian GLP-1 (nmGLP-1) peptides and analogs thereof disclosed herein and in particular SEQ ID NOS: 6-47 and SEQ ID NOS: 53-56. The nmGLP-1 analogs of the present invention will generally retain, at least in part, a biological activity similar to that of native non-mammalian GLP-1, i.e., the nmGLP-1 analogs of the present invention will generally have GLP-1-like activity and in particular are effective in reducing food intake and/or appetite. Thus in one embodiment is provided a non-mammalian GLP-1 (nmGLP-1) analog peptide said nmGLP-1 analog peptide comprising an amino acid sequence selected from at least one of the group consisting of SEQ ID NO: 6 to SEQ ID NO: 47 and SEQ ID NO: 53 to SEQ ID NO: 56. In certain embodiments, the nmGLP-1 peptide is an isolated peptide.

Another embodiment provides an isolated non-mammalian GLP-1 (nmGLP-1) analog peptide said nmGLP-1 analog peptide consisting essentially of an amino acid sequence selected from at least one of the group consisting of SEQ ID NO: 6 to SEQ ID NO: 47 and SEQ ID NO: 53 to SEQ ID NO: 56. In certain embodiments, the nmGLP-1 peptide is an isolated peptide. Yet another embodiment provides a non-mammalian GLP-1 (nmGLP-1) analog peptide said nmGLP-1 analog peptide consisting of an amino acid sequence selected from at least one of the group consisting of SEQ ID NO: 6 to SEQ ID NO: 47 and SEQ ID NO: 53 to SEQ ID NO: 56. In particular embodiments, the nmGLP-1 peptide is an isolated peptide.

In a further embodiment is provided an isolated non-mammalian GLP-1 (nmGLP-1) analog peptide said nmGLP-1 analog peptide comprising of an amino acid sequence having at least 90% sequence identity to a polypeptide selected from at least one of the group consisting of SEQ ID NO: 6 to SEQ ID NO: 47 and SEQ ID NO: 53 to SEQ ID NO: 56; wherein said polypeptide is not a mammalian GLP-1 or an exendin and in particular is not any one of, any subgroup of, or all of SEQ ID NOS: 1-5 or 48-52. In certain embodiments, the nmGLP-1 peptide is an isolated peptide.

Another aspect, provides pharmaceutical compositions comprising a non-mammalian GLP-1 or non-mammalian GLP-1 analog and a pharmaceutically acceptable vehicle or carrier. In one embodiment, the non-mammalian GLP-1 or non-mammalian GLP-1 analog is selected from at least one of the group consisting of SEQ ID NOS: 1, 2, and 6-56. In another embodiment, the non-mammalian GLP-1 or non-mammalian GLP-1 analog is selected from at least one of the group consisting of SEQ ID NOS: 6-47 and SEQ ID NOS: 53-56.

Yet another aspect provides methods of treating a subject, for example a mammal and more particularly a human, having a condition for which administration of a GLP-1 compound is indicated, where the method comprises administering a pharmacologically effective amount of an nmGLP-1 or analog thereof and in particular the nmGLP-1 peptides and analogs thereof selected from at least one of the group consisting of SEQ ID NOS: 1, 2, and 6-56. In another embodiment, the non-mammalian GLP-1 or non-mammalian GLP-1 analog is selected from at least one of the group consisting of SEQ ID NOS: 6-47 and SEQ ID NOS: 53-56.

Yet a further aspect provides methods for controlling appetite in a subject, for example a mammal and more particularly a human, in need thereof comprising administering an effective amount of a non-mammalian GLP-1 (nmGLP-1) or analog thereof, for example, the nmGLP-1 peptides and analogs described herein and in particular any one or more of SEQ ID NOS: 1, 2, and 6-56. In another embodiment, the non-mammalian GLP-1 or non-mammalian GLP-1 analog is selected from at least one of the group consisting of SEQ ID NOS: 6-47 and SEQ ID NOS: 53-56.

Still a further aspect provides methods for treating obesity in a subject, for example a mammal and more particularly a human, in need thereof comprising administering to a patient an effective amount of nmGLP-1 or analog thereof, for example, the nmGLP-1 peptides and analogs described herein and in particular one or more of SEQ ID NOS: 1, 2, and 6-56. In another embodiment, the non-mammalian GLP-1 or non-mammalian GLP-1 analog is selected from at least one of the group consisting of SEQ ID NOS: 6-47 and SEQ ID NOS: 53-56.

Another aspect, the present invention provides methods for reducing food intake in a subject in need thereof comprising administering an effective amount of an nmGLP-1 or analog thereof, for example, the nmGLP-1 peptides and analogs described herein, and in particular any one or more selected from the group consisting of SEQ ID NOS: 1, 2 and 6-56. In another embodiment, the non-mammalian GLP-1 or non-mammalian GLP-1 analog is selected from at least one of the group consisting of SEQ ID NOS: 647 and SEQ ID NOS: 53-56.

Another embodiment of the present application comprises a method for reducing food intake in a mammal, for example, a human, the method comprising administering at least one naturally-occurring nmGLP-1 peptide. Yet another embodiment of the present application comprises a method for reducing food intake in a mammal, for example, a human, the method comprising administering at least one analog or agonist of a naturally-occurring nmGLP-1 peptide. In one embodiment, the nmGLP-1 peptide or nmGLP-1 peptide analog is at least one selected from the group consisting of SEQ ID NO: 1, 2 and 6-56. In another embodiment, the non-mammalian GLP-1 or non-mammalian GLP-1 analog is selected from at least one of the group consisting of SEQ ID NOS: 6-47 and SEQ ID NOS: 53-56.

Another embodiment of the present application comprises a method for controlling appetite in a mammal, for example a human, the method comprising administering at least one naturally-occurring nmGLP-1 peptide. Yet another embodiment of the present application comprises a method for controlling appetite in a mammal, for example a human, the method comprising administering at least one analog or agonist of a naturally-occurring nmGLP-1 peptide. In one embodiment the nmGLP-1 or nmGLP-1 analog is at least one selected from the group consisting of SEQ ID NOS: 1, 2, and 6-56. In another embodiment, the non-mammalian GLP-1 or non-mammalian GLP-1 analog is selected from at least one of the group consisting of SEQ ID NOS: 6-47 and SEQ ID NOS: 53-56.

Another embodiment of the present application comprises a method for obtaining or maintaining weight reduction in a mammal, for example, a human, the method comprising administering at least one naturally-occurring nmGLP-1 peptide. Yet another embodiment of the present application comprises a method for obtaining or maintaining weight reduction in a mammal, for example, a human, the method comprises administering at least one analog or agonist of a naturally-occurring nmGLP-1 peptide. In one embodiment the nmGLP-1 or nmGLP-1 analog is at least one selected from the group consisting of SEQ ID NOS: 1, 2, and 6-56. In another embodiment, the non-mammalian GLP-1 or non-mammalian GLP-1 analog is selected from at least one of the group consisting of SEQ ID NOS: 647 and SEQ ID NOS: 53-56.

In any of the preceding methods, the nmGLP-1 or nmGLP-1 analog can be administered by a single intravenous injection, continuous intravenous administration, single subcutaneous injection, continuous subcutaneous administration, a regimen of multiple subcutaneous injections, micropressure injection system, ambulatory pump, depot sustained-release injection, implant, deep lung sustained-release insufflation, skin patch, buccal patch and a sustained-release oral delivery dose form.

Any of these methods can be accomplished in the following general manner: identifying a patient in need, determining a therapeutic dose to achieve the appetite control, weight reduction, decrease in food intake, etc. desired, administering the therapeutic dose in a manner suitable to achieve the desired result(s), monitoring the patient in need to determine if the desired result(s) are being achieved and make necessary changes, weaning the patient from the therapeutic dose or if necessary maintaining the patient on the therapeutic dose. The methods can use some or all of these steps, and can do so in any order. The methods and compositions of the present invention can be used with a patient in need thereof.

In another embodiment, the methods outlined further comprise the identification of a subject in need of treatment. Any effective criteria can be used to determine that a subject can benefit from administration of an nmGLP-1 peptide or analogue thereof. Methods for the diagnosis of obesity, for example, as well as procedures for the identification of individuals at risk for development of these conditions, are well known to those in the art. Such procedures can include clinical tests, physical examination, personal interviews and assessment of family history.

Also provided is method for stimulating insulin secretion in a mammal comprising the step of administering to the mammal, for example a human, an amount of an nmGLP-1 or analog thereof which is effective to stimulate insulin secretion. In certain embodiments the mammal suffers from impaired glucose tolerance, insulin resistance, type I diabetes, type II diabetes or gestational diabetes. In particular embodiments, the nmGLP-1 or analog thereof is at least one selected from the group consisting of SEQ ID NOS: 1, 2 and 6-46. In another embodiment, the nmGLP-1 or analog thereof is at least one selected from the group consisting of SEQ ID NOS: 6-47 and 53-56.

One aspect provides peptide, for example an nmGLP-1 peptide or analog thereof, that has greater stability as compared to a mammalian GLP-1 peptide by the addition of any one of SEQ ID NOS: 57-68, particularly to the C terminal end of said mammalian peptide. Another aspect provides a method for increasing the stability of a peptide, especially to enzymatic degradation such as takes place in blood or serum or plasma by the addition of any one of SEQ ID NOS: 57-68, particularly to the C terminal end of said mammalian peptide.

Also provided are methods for treating a human afflicted with a condition selected from the group consisting of non-insulin-dependent type II diabetes, obesity and type I diabetes, comprising the step of administering to the human an amount of an nmGLP-1 or analog thereof, for example, the nmGLP-1 peptides and analogs described herein, in particular SEQ ID NO. 1, 2 and 6-56, which is effective for alleviation of said condition. In another embodiment, the nmGLP-1 or analog thereof is at least one selected from the group consisting of SEQ ID NOS: 6-47 and 53-56.

Also provided are methods of treating diseases conditions or disorders, wherein said diseases, conditions or disorders can be treated or alleviated by administration of a GLP-1 peptide, by administering an nmGLP-1 peptide or analog thereof effective to treat or alleviate said disease, condition or disorder. In particular embodiments, the nmGLP-1 or analog thereof is at least one selected from the group consisting of SEQ ID NOS: 1, 2 and 6-46. In another embodiment, the nmGLP-1 or analog thereof is at least one selected from the group consisting of SEQ ID NOS: 6-47 and 53-56. For example, the peptides and pharmaceutical compositions of the present invention can be used to treat irritable bowel syndrome (see WO 99/64060), or to reduce the morbidity and mortality associated with myocardial infarction (see WO 93/08531) and stroke (see WO 00/16797) as well as catabolic changes that occur after surgery (see U.S. Pat. No. 6,006,753), or related conditions in mammals such as humans, and to reduce the mortality and morbidity that occurs in critically ill patients that experience respiratory distress or have illness or condition that is likely to lead to respiratory distress. Examples of conditions that involve respiratory distress include acute lung injury, respiratory distress syndrome, cor pulmonale, chronic obstructive pulmonary disease, and sepsis. Also included are subjects at risk for developing non-insulin dependent diabetes (see WO 00/07617), such as those with prediabetes.

### DETAILED DESCRIPTION

The term “effective amount” refers to an amount of an agent, for example a pharmaceutical agent, used to treat, ameliorate, prevent, or eliminate the identified condition (e.g., disease or disorder), or to exhibit a detectable therapeutic, preventative or physiological effect. The effect can be detected by, for example, chemical markers, antigen levels, or time to a measurable event, such as food intake or weight loss. The precise effective amount for a subject will depend upon the subject's body weight, size, and health; the nature and extent of the condition; and the therapeutic or combination of therapeutics selected for administration. Effective amounts for a given situation can be determined by routine experimentation that is within the skill and judgment of the clinician.

In particular embodiments, a “subject in need thereof” is a subject who is afflicted with or has an undesirable or deleterious symptom associated with one of the diseases, conditions or disorders described herein, for example is overweight or obese, or who is in need of reducing food or caloric intake, or both overweight or obese and in need of reducing food or caloric intake. As used herein, a “desirous” subject is a subject who wishes to reduce their body weight or food intake or wishes to reduce both their body weight and food intake regardless of if they are medically considered to be overweight or obese, for example, in order to reduce, treat, prevent or delay the onset of at least one of the symptoms or afflictions associated with a disease, condition or disorder described herein. In one embodiment, the subject is an obese or overweight subject. In one aspect, an “overweight subject” refers to a subject with a body mass index (BMI) greater than or equal to 25, for example, a BMI between 25 and 30. While “obesity” is generally defined as a body mass index of 30 or greater, for certain embodiments, any subject who needs or desires to reduce body weight is included in the scope of “obese.” In one aspect, subjects who are insulin resistant, glucose intolerant, or have any form of diabetes mellitus (e.g., type 1, 2 or gestational diabetes) can benefit from the administration of the nmGLP-1 peptides and analogs disclosed herein, and in particular any one of SEQ ID NOS: 1, 2 and 6-56 or in a further embodiment SEQ ID NOS: 6-47 and SEQ ID NOS: 53-56. In another aspect, the subject suffering from insulin resistance, glucose intolerance or diabetes mellitus is also obese or overweight.

The term “amino acid” refers to natural amino acids, unnatural amino acids, and amino acid analogs, all in their D and L stereoisomers if their structures allow such stereoisomeric forms. Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine. N-methylvaline, naphthalanine, norvaline, norleucine, octylglycine, ornithine, pentylglycine, pipecolic acid and thioproline. Amino acid analogs include the natural and unnatural amino acids which are chemically blocked, reversibly or irreversibly, or modified on their N-terminal amino group or their side-chain groups, as for example, methionine sulfoxide, methionine sulfone, S-(carboxymethyl)-cysteine, S-(carboxymethyl)-cysteine sulfoxide and S-(carboxymethyl)-cysteine sulfone.

The term “amino acid analog” refers to an amino acid where the C-terminal carboxy group, the N-terminal amino group or side-chain functional group has been chemically modified to another functional group. For example, aspartic acid-(beta-methyl ester) is an amino acid analog of aspartic acid. N-ethylglycine is an amino acid analog of glycine; or alanine carboxamide is an amino acid analog of alanine. The term “alkyl” as used herein means a straight or branched aliphatic hydrocarbon group. Non limiting examples of substituents are straight or branched alkyl such as methyl, ethyl, propyl, isopropyl, butyl, isobutyl, sec-butyl, pentyl, hexyl; and alkenyl, hydroxyl, alkoxy, amino, halo.

As used herein, the term “acylation” refers to the chemical transformation which substitutes an acyl (RCO—) group into a molecule, generally for an active hydrogen of an —OH group. The term “acyl” denotes a radical provided by the residue after removal of hydroxyl from an organic acid. Examples. of such acyl radicals include alkanoyl and aroyl radicals. Examples of lower alkanoyl radicals include, but are not limited to, formyl, acetyl, propionyl, butyryl, isobutyryl, valeryl, isovaleryl, pivaloyl, hexanoyl, trifluoroacetyl.

The term “lower” referred to herein in connection with organic radicals such as allyl or acyl groups defines such groups with up to and including about six, up to and including four or one or two carbon atoms. Such groups can be straight chains or branched chains.

The term “pharmaceutically acceptable salt” includes salts of the compounds described herein derived from the combination of such compounds and an organic or inorganic acid. In practice the use of the salt form amounts to use of the base form. The compounds are useful in both free base and salt forms. The term “pharmaceutically acceptable excipient” refers to an excipient for administration of a pharmaceutical agent. The term refers to any pharmaceutical excipient that can be administered without undue toxicity. Pharmaceutically acceptable excipients are determined in part by the particular composition being administered, as well as by the particular method used to administer the composition. Accordingly, there exists a wide variety of suitable formulations of pharmaceutical compositions for use in the methods of the present application (see, e.g., Remington's Pharmaceutical Sciences).

As used herein, the term “hGLP-1” refers to human GLP-1 (SEQ ID NO: 3) which is specifically excluded from the definition of nmGLP-1.

“nmGLP-1” refers to a non-mammalian GLP-1 peptide and unless otherwise indicated specifically excludes known exendins, for example, exendins 1-4, and in particular SEQ ID NOS: 4 and 5.

“CAN” or “CH₃CN” refers to acetonitrile.

“Abu” means 2-Aminobutyric acid.

“Boc”, “tBoc” or “Tboc” refers to t-butoxy carbonyl.

“Aib” means 2-Aminoisobutyric acid.

“Fmoc” refers to fluorenylmethoxycarbonyl.

“HBTU” refers to 2-(1H-benzotriazol-1-yl)-1,1,3,3,-tetramethyluronium hexafluorophosphate.

“HOBt” refers to 1-hydroxybenzotriazole monohydrate.

Isocap refers to isocaproic acid or 4-methyl pentanoic acid

With the exclusion of SEQ ID NO: 3, Table 1 provides non-mammalian glucagon-like peptide-1 (nmGLP-1) peptides and biologically active analogs of nmGLP-1. nmGLP-1 peptides and analogs thereof, particularly agonist analogs, have been found to be effective in suppressing appetite and reducing the urge to intake food. Multiple analogs of nmGLP-1 for use in the methods and composition provided herein are disclosed, for example, SEQ ID NOS: 6-47, 53-68. In one embodiment, nmGLP-1 peptides do not include naturally occurring nmGLP-1 peptides for example SEQ ID NOS: 1, 2, 4, 5, and 48-52. In another embodiment the nmGLP-1 peptides do not include SEQ ID NO 1-5.

Another embodiment relates to nmGLP-1 peptides and analogs of nmGLP-1 peptides, for example, SEQ ID NOS: 1, 2, and 6-56; SEQ ID NOS: 6-56; or SEQ ID NOS: 6-47 and 53-56, with a pharmaceutically acceptable carrier to provide an effective treatment composition for mammals, for example humans. The treatment composition can used, for example, in methods of preventing or reducing obesity, reducing or controlling appetite, reducing food intake, or reducing food intake and controlling appetite in a mammal, for example a human.

TABLE 1

| SEQ ID NO | Sequence |
|---|---|
| 1 | HAEGTFTSDVTQQLDEKAAKEFIDWLINGGPSKEIIS |
| 2 | HAEGTYTNDVTEYLEEKAAKEFIEWLIKGKPKKIRYS |
| 3 | HAEGTFTSDVSSYLEGQAADEFIAWLVKGR |
| 4 | HGEGTFTSDLSKQMEEEAVRLFIEWLKNGGPSSGAPPPS |
| 5 | HGEGTFTSDLSKQMEEEAVRLFIEWLKN |
| 6 | HAEGTFTNDMTNYLEEKAAKEFVGWLIKGRS |
| 7 | HAEGTFTSDVTQQLDEKAAKEFIDWLIN |
| 8 | HaEGTFTSDVTQQLDEKAAKEFIDWLIN |
| 9 | HAEGTFTSDVTQQLDEKAAKEFIDWLINGGPSSGAPPPS |
| 10 | HaEGTFTSDVTQQLDEKAAKEFIDWLINGGPSKEIIS |
| 11 | HAEGTFTSDVTQQLDEKAAKEFIDWLINGGPSKEIIS |
| 12 | HAEGTFTSDVTQQLDEKAAKEFIDWLINGGASKEIIS |
| 13 | HAEGTFTSDVTQQLDEKAAKEFIDALINGGPSKEIIS |
| 14 | HAEGTFTSDVTQQLDEKAAKEFIDWLINPGSEGIKSI |
| 15 | HaEGTFTSDVTQQLDEKAAKEFIDWLINGGPSKEIIK |
| | (isocap) |
| 16 | HaEGTFTSDVTQQLDEKAAK(isocap)EFIDWLINGGPSK |
| | EIIS |
| 17 | HaEFTFTSDVTQQLDEKAAC(n-nonyl acetamide)EF |
| | IDWLINGGPSKEIIS |
| 18 | HaEFTFTSDVTQQLDEKAA(Octylglycine)EFIDWLIN |
| | GGPSKEIIS |
| 19 | HAEFTYTNDVTEYLEEKAAKEFIEWLIKGGPSSGAPPPS |
| 20 | HaEGTYTNDVTEYLEEKAAKEFIEWLIKGGPSSGAPPPS |
| 21 | HAEGTYTNDVTEYLEEKAAKEFIEWLINGGPSKEIIS |
| 22 | HAEGTFTSDVTQQLEEKAAKEFIEWLIKGKPKKIRYS |
| 23 | HaEGTYTNDVTEYLEEKAAKEFIEWLIKGKPKKIRYS |
| 24 | HAEGTYTNDVTEYLEEKAAKEFIEWLIN |
| 25 | HAEGTYTNDVTEYLEEKAAKEFIEWLIK |
| 26 | HGEGTYTNDVTEYLEEKAAKEFIEWLIKGKPKKIRYS |
| 27 | H(Aib)EGTYTNDVTEYLEEKAAKEFIEWLIKGKPKKIRYS |
| 28 | H(Abu)EGTYTNDVTEYLEEKAAKEFIEWLIKGKPKKIRYS |
| 29 | HAEGTYTNDVTEYLEKAAKEFIEWLIKGKPKKIRYS |
| 30 | HAEGTYTNDVTEYLEEAAKEFIEWLIKGKPKKIRYS |
| 31 | HAEGTYTNDVTEYLEEKAAKFIEWLIKGKPKKIRYS |
| 32 | HAEGTYTNDVTEYLEEKAAKEFIEWLIPKKIRYS |
| 33 | HAEGTYTNDVTELEEKAAKEFIEWLIKGKPKKIRYS |
| 34 | HAEGTYTNDVTEYLEEKAAKEFIEWLIKGKPKKIRY |
| 35 | HAEGTYTNDVTEYLEEKAAKEFIEWLIGKPKKIRYS |
| 36 | HAEGTYTNDVTEYLEEKAAKEFIEWLIKG-PKKIRYS |
| 37 | EGTYTNDVTEYLEEKAAKEFIEWLIKGKPKKIRYS |
| 38 | HAEGTYTNDVTEYEEKAAKEFIEWLIKGKPKKIRYS |
| 39 | HAEGTYTNDTEYLEEKAAKEFIEWLIKGKPKKIRYS |
| 40 | HAEGTYTNDVTEYLEEKAAKEFIEWLIKGKPK |
| 41 | HAEGTYTNDVTYLEEKAAKEFIEWLIKGKPKKIRYS |
| 42 | HAEGTYTNDVTEYLEEKAAKEFIEWLI |
| 43 | HaEGTYTNDVTEYLEEKAAKEFIEWLI |
| 44 | HAEGTYTNDVTEYLEEKAAKEFIEWL |
| 45 | HaEGTYTNDVTEYLEEKAAKEFIEWL |
| 46 | HAEGTYTNDVTEYLEEKAAKEFIEW |
| 47 | HaEGTYTNDVTEYLEEKAAKEFIEW |
| 48 | HADGTYTSDVSSYLQDQAAKDFITWLKSGQPKPDAVET |
| 49 | HADGTYTSDVSSYLQDQAAKDFITWLKSGQPKPDAVETRSV |
| | DTL |
| 50 | HAEGTYTNDVTQFLEEKAAKEFIDWLIKGKPKKQRLS |
| 51 | YADAPYISDVYSYLQDQVAKKWLKSGQD |
| 52 | HADGTFTNDMTSYLDAKAARDFVSWLARSDKS |
| 53 | H(Abu)EGTYTNDVTQFLEEKAAKEFIDWLIKGKPKKQRLS |
| 54 | HaEGTYTNDVTQFLEEKAAKEFIDWLIKGKPKKQRLS |
| 55 | HGEGTYTNDVTQFLEEKAAKEFIDWLIKGKPKKQRLS |
| 56 | H(Aib)EGTYTNDVTQFLEEKAAKEFIDWLIKGKPKKQRLS |

a = dAla

The nmGLP-1 peptides or analogs thereof of the present application include those disclosed in Table 1. It should be appreciated that any of the peptides disclosed herein and in particular those disclosed in Tables 1 and 2 can exist and be used in either the acid (OH) or amide (NH₂) forms. The term “analog” as used herein means non-naturally occurring nmGLP-1 peptides. Examples of nmGLP-1 analogs are set forth in Table 1, with the exception of SEQ ID NO: 3 which is a human GLP-1. Specific analogs include SEQ ID NOS: 6-47 and 53-56 or SEQ ID NOS: 7-47 and 53-56. Activity of nmGLP-1 peptides or analogs thereof can be determined by, for example, the assays described herein. Effects of nmGLP-1 peptides or analogs thereof on preventing or reducing obesity, reducing body weight, controlling or reducing appetite, reducing food intake, or reducing food intake, and controlling appetite in a mammal can be identified, evaluated, or screened for, using the methods described in the examples herein, or other methods known in the art.

Functional variants of nmGLP-1 peptides or analogs thereof are also included. Functional variants of nmGLP-1 peptides or analogs thereof include those that retain, to some extent, the receptor binding, food intake lowering, stimulation of insulin secretion or other activities of the related nmGLP-1 peptide or analog thereof. A functional variant has an activity that can be substituted for one or more activities of a particular nmGLP-1 peptide or analog thereof. In one embodiment functional variants retain all of the activities of a particular nmGLP-1 peptide or analog thereof.

In another embodiment, the functional variant can have an activity that, when measured quantitatively, is stronger or weaker, as measured in functional assays, for example, such as those disclosed herein. Exemplary functional variants have activities that are within about 10% to about 500% of the activity of an nmGLP-1 peptide or analog thereof disclosed in Table 1, between about 5% to about 250%, and within about 5% to about 100%. Exemplary functional variants include those that have activities that, when measured quantitatively, are stronger or weaker, as measured in functional assays, for example, such as those disclosed herein, greater than about 5, 10, or 25% of an nmGLP-1 or an analog disclosed in Table 1. In some embodiments the functional variants have not more that 5, 4, 3, or 2 amino acid substitutions, deletions or additions as compared to the reference molecule, for example, a naturally occurring non-mammalian GLP-1 described herein. In other embodiments, the functional variants have not more than 1 amino acid substitution, deletion, or addition. In certain embodiments, the nmGLP-1 having not more than 5 amino acid substitutions, additions or deletions is not a mammalian GLP-1 or an exendin and in particular is not any one of SEQ ID NOS: 3-5. In another embodiment, the nmGLP-1 having not more than 5 amino acid substitutions, additions or deletions is not any one of, any subgroup of, or all of SEQ ID NOS: 1-5 and 48-52.

In certain embodiments the nmGLP-1 peptides useful in the compositions, methods and uses disclosed herein, include those nmGLP-1 peptides listed in Table 4 which show a percent inhibition of food intake of at least 50% for any time period shown, i.e. 30, 60, 120 or 180 minutes, and are not SEQ ID NOS: 3-5. In another embodiment, nmGLP-1 peptides useful in the compositions, methods and uses disclosed herein, include those nmGLP-1 peptides listed in Table 4 which show a percent inhibition of food intake of at least 25% for any time period shown and are not SEQ ID NOS: 3-5. In yet another embodiment, nmGLP-1 peptides useful in the compositions, methods and uses disclosed herein, include those nmGLP-1 peptides listed in Table 4 which show a percent inhibition of food intake of at least 15% for any time period shown and are not SEQ ID NOS: 3-5. In still another embodiment, nmGLP-1 peptides useful in the compositions, methods and uses disclosed herein, include those nmGLP-1 peptides listed in Table 4 which show a percent inhibition of food intake of at least 10% for any time period shown and are not SEQ ID NOS: 3-5. In a further embodiment, nmGLP-1 peptides useful in the compositions, methods and uses disclosed herein, include those nmGLP-1 peptides listed in Table 4 which show a percent inhibition of food intake of at least 5% for any time period shown and are not SEQ ID NOS: 3-5. Thus, in certain embodiments, the compositions and methods for inhibiting food intake, reducing appetite and/or treating obesity exclude SEQ ID NOS: 29, 31, 37, 39, 41, 46, 47 and 51.

In certain embodiments the nmGLP-1 peptides useful in the compositions, methods and uses disclosed herein, include those nmGLP-1 peptides listed in Table 4 which show a percent inhibition of food intake of at least 50% for all time periods shown, i.e. 30, 60, 120 and 180 minutes, and are not SEQ ID NOS: 3-5. In another embodiment, nmGLP-1 peptides useful in the compositions, methods and uses disclosed herein, include those nmGLP-1 peptides listed in Table 4 which show a percent inhibition of food intake of at least 25% for all time periods shown and are not SEQ ID NOS: 3-5. In yet another embodiment, nmGLP-1 peptides useful in the compositions, methods and uses disclosed herein, include those nmGLP-1 peptides listed in Table 4 which show a percent inhibition of food intake of at least 15% for all time periods shown and are not SEQ ID NOS: 3-5. In still another embodiment, nmGLP-1 peptides useful in the compositions, methods and uses disclosed herein, include those nmGLP-1 peptides listed in Table 4 which show a percent inhibition of food intake of at least 10% for all time periods shown and are not SEQ ID NOS: 3-5. In a further embodiment, nmGLP-1 peptides useful in the compositions, methods and uses disclosed herein, include those nmGLP-1 peptides listed in Table 4 which show a percent inhibition of food intake of at least 5% for all time periods shown and are not SEQ ID NOS: 3-5.

An nmGLP-1 peptide or analog thereof can be modified. Such modifications include, but are not limited to, phosphorylation, glycosylation, crosslinking, acylation, alkylation, proteolytic cleavage, linkage to an antibody molecule, membrane molecule or other ligand. An nmGLP-1 peptide or nmGLP-1 analog can be carboxy-terminal amidated or hydroxy form. An nmGLP-1 analog can be an agonist.

The term “agonist” refers to a molecule that has an affinity for a receptor associated with the reference molecule and stimulates at least one activity associated with the reference molecule binding to that receptor. For example, and without limitation, an nmGLP-1 agonist binds to a receptor that also binds a GLP-1 peptide, for example an nmGLP-1 peptide, and as a result of the agonist binding stimulates at least one activity associated with the binding of the GLP-1 that naturally binds to the same receptor. In specific embodiments, the GLP-1 receptor agonist is an analog of a naturally occurring nmGLP-1 peptide, for example SEQ ID NOS: 6-47 and 53-56.

nmGLP-1 or nmGLP-1 analogs can also be further derivatized by chemical alterations such as amidation, glycosylation, acylation, sulfation, phosphorylation, acetylation, and cyclization. Such chemical alterations can be obtained through chemical or biochemical methodologies, as well as through in-vivo processes, or any combination thereof. Derivatives of the nmGLP-1 or nmGLP-1 analog peptides disclosed herein can also include conjugation to one or more polymers or small molecule substituents. One type of polymer conjugation is linkage or attachment of polyamino acids (e.g., poly-his, poly-arg, poly-lys, etc.) and/or fatty acid chains of various lengths to the N- or C-terminus or amino acid residue side chains of an nmGLP-1 or nmGLP-1 analog. Small molecule substituents include short alkyls and constrained alkyls (e.g. branched, cyclic, fused, adamantyl), and aromatic groups.

In one embodiment, nmGLP-1 peptides or analogs thereof can be coupled to polyethylene glycol (PEG) by one of several strategies. Those skilled in the art will be able to utilize well-known techniques for linking one or more PEG polymers to nmGLP-1 and/or nmGLP-1 analogs, described herein. Suitable PEG polymers typically are commercially available or can be made by techniques well know to those skilled in the art. In one embodiment, the polyethylene glycol polymers have molecular weights between 500 and 20,000 and can be branched or straight chain polymers. In other embodiments, the nmGLP-1 peptides or analogs thereof are modified by the addition of polyamide chains of precise lengths as described, for example, in U.S. Pat. No. 6,552,167, the content of which is incorporated by reference in its entirety. In other embodiments, the nmGLP-1 peptides or agonists or analogs thereof are modified by the addition of alkylPEG moieties as described in U.S. Pat. Nos. 5,359,030 and 5,681,811, the contents of which are incorporated by reference in their entirety.

An attachment of a PEG on an intact peptide or protein can be accomplished by coupling to amino, carboxyl or thiol groups. Such groups will typically be the N and C termini and on the side chains of such naturally occurring amino acids as lysine, aspartic acid, glutamic acid and cysteine. Since the compounds of the present invention can be prepared by solid phase peptide chemistry techniques, a variety of moieties containing diamino and dicarboxylic groups with orthogonal protecting groups can be introduced for conjugation to PEG.

An nmGLP-1 peptide or analog thereof can be linked to one or more macromolecules other than polyethylene glycol. Examples of such macromolecules include albumin, gelatin and antibodies. When the macromolecule is an antibody it can be a single chain antibody, an intact antibody or a fragment of an antibody, such as an Fc or Fab fragment. In a particular embodiment, the intact antibody is a catalytic antibody and the nmGLP-1 is attached at the antibody's catalytic site via an appropriate hapten linker.

nmGLP-1 peptides or analogs thereof can be synthesized by any method, or combination of methods, including chemical and biological. nmGLP-1 peptides or analogs thereof can also be synthesized in vitro or in vivo, or a combination of in vitro and in vivo methods. Chemical methods include automated and manual peptide synthesis. Biological methods conventional recombinant techniques described in detail for example in Sambrook et al., Molecular Cloning: A Laboratory Manual, 2d Ed., Cold Spring Harbor (1989). “Recombinant”, as used herein, means that a peptide is derived from recombinant (e.g., microbial or mammalian) expression systems. Other compounds useful in the present application can be prepared by art-known methods. For example, phosphate-containing amino acids and peptides containing such amino acids, can be prepared using methods known in the art.

Standard solid-phase peptide synthesis techniques, for example, an automated or semiautomated peptide synthesizer can be used. Typically, using such techniques, an α-N-carbamoyl protected amino acid and an amino acid attached to the growing peptide chain on a resin are coupled at room temperature in an inert solvent such as dimethylformamide, N-methylpyrrolidinone or methylene chloride in the presence of coupling agents such as dicyclohexylcarbodiimide and 1-hydroxybenzotriazole in the presence of a base such as diisopropylethylamine. The α-N-carbamoyl protecting group is removed from the resulting peptide-resin using a reagent such as trifluoroacetic acid or piperidine, and the coupling reaction repeated with the next desired N-protected amino acid to be added to the peptide chain. Suitable N-protecting groups are well known in the art, with tBoc and Fmoc being typically used.

Solid phase peptide synthesis can be carried out with an automatic peptide synthesizer (Model 430A, Applied Biosystems Inc., Foster City. Calif.) using the NMP/HOBt (Option 1) system and tBoc or Fmoc chemistry with capping. Boc-peptide-resins can be cleaved with HF (−5° C. to 0° C., 1 hour). The peptide can be extracted from the resin with alternating water and acetic acid, and the filtrates lyophilized. Fmoc-peptide resins can be cleaved according to standard methods. Peptides can also be assembled using an Advanced Chem Tech Synthesizer (Model MPS 350, Louisville. Ky.).

nmGLP-1 peptides or analogs thereof can be a naturally purified product, or a product of chemical synthetic procedures, or produced by recombinant techniques from prokaryotic or eukaryotic hosts (for example by bacterial, yeast, higher plant, insect and mammalian cells in culture). Depending on the host employed in a recombinant production procedure, the peptides of the present application can be glycosylated or can be non-glycosylated. nmGLP-1 peptides or analogs thereof of the application can also include an initial methionine amino acid residue.

nmGLP-1 peptides or analogs thereof can be produced in full-length form or in partial-length sequences, which can be assembled in vitro or in vivo. In one embodiment, the nmGLP-1 peptides, agonists, or analogs thereof are synthesized using chemical methods, more specifically using an automated peptide synthesis machine, for example, solid-state direct amino acid synthesis.

nmGLP-1 peptides, agonists, or analogs thereof can be isolated and purified using any means in order to achieve the desired purity. Methods of isolation and purification include: ammonium sulfate or ethanol precipitation, salting out, acid extraction, phosphocellulose chromatography, hydrophobic interaction chromatography, hydroxylapatite chromatography, lectin chromatography, gel-filtration chromatography, ion (anion or cation)-exchange chromatography, affinity chromatography, high-performance liquid chromatography (HPLC), column chromatography, flash chromatography, gel electrophoresis, dialysis, precipitation, His-tag protein purification, isoelectric focusing, and two-dimensional electrophoresis. HPLC can be employed for final purification steps.

Peptides can be purified by RP-HPLC (preparative and analytical) using a Gilsons preparative HPLC system. A C4, C8 or C18 preparative column (10μ, 2.2×25 cm; Vydac, Hesperia, Calif.) can be used to isolate peptides, and purity can be determined using a C4, C8 or C18 analytical column (5μ, 0.46×25 cm; Vydac). Solvents (A=0.1% TFA/water and B=0.1% TFA/CH₃CN) can be delivered to the analytical column at a flow rate of 1.0 ml/min and to the preparative column at 15 ml/min. Amino acid analyses can be performed on the Waters Pico Tag system and processed using the Maxima program. Peptides can be hydrolyzed by vapor-phase acid hydrolysis (115° C., 20 to 24 h). Hydrolysates can be derivatized and analyzed by standard methods. Fast atom bombardment analysis can be carried out by M-Scan, Incorporated (West Chester, Pa.). Mass calibration can be performed using cesium iodide or cesium iodide/glycerol. Plasma desorption ionization analysis using time of flight detection can be carried out on an Applied Biosystems Bio-Ion 20 mass spectrometer. Electrospray mass spectroscopy can be carried out on a VG-Trio machine.

Activity of nmGLP-1 peptides or analogs thereof can be determined by standard methods, in general, by receptor-binding activity screening procedures involve providing appropriate cells which express the receptor on the surface thereof, for example insulinoma cell lines such as RINmSF cells or INS-1 cells. In addition to measuring specific binding of tracer to membrane, cAMP activity or glucose dependent insulin production can also be measured. In one method, a polynucleotide encoding a GLP-1 receptor, for example a nmGLP-1 receptor, is employed to transfect cells to express a functional GLP-1 receptor. Thus, for example, such assay can be employed for screening for a receptor agonist by contacting such cells with compounds to be screened and determining whether such compounds generate a signal, i.e., activate the receptor.

Other screening techniques include the use of cells which express a GLP-1 peptide receptor, for example a nmGLP-1 receptor, such as transfected CHO cells in a system which measures extracellular pH or ionic changes caused by receptor activation. For example, potential agonists can be contacted with a cell which expresses the nmGLP-1 peptide receptor and a second messenger response, e.g., signal transduction or ionic or pH changes, can be measured to determine whether the potential agonist is effective.

An embodiment comprises nmGLP-1 peptide, agonist, or analog thereof sequences that show about 90% to about 97% identity to the sequences shown in Table 1, for example, sequences that show about 92% to about 97% identity, or sequences that show about 95% or greater identity. Such sequences do not include mammalian GLP-1 peptides and exendins and in particular do not include SEQ ID NOS: 3-5. In other embodiments, such sequences further do not include any one of, any subgroup of, or all of SEQ ID NOS: 1, 2 and 48-52. Sequences that differ from the peptide sequences in Table 1 by any one of one, two, three, four, or five amino acids and do not include any one, any subgroup or all SEQ ID NOS: 1-5 and 48-52, as well as any mammalian GLP-1 or exendin are also provided.

Sequence identity, as is well understood in the art, is a relationship between two or more polypeptide sequences or two or more polynucleotide sequences, as determined by comparing the sequences. In the art, identity can also mean the degree of sequence relatedness between polypeptide or polynucleotide sequences, as determined by the match between strings of such sequences. Identity can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J Applied Math, 48:1073 (1988). Methods to determine identity are designed to give the largest match between the sequences tested. Moreover, methods to determine identity are codified in publicly available programs. Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al, Genome Analysis, 1: 543-559 (1997)). The BLAST X program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). The well known Smith Waterman algorithm can also be used to determine identity.

Parameters for polypeptide sequence comparison typically include the following: Algorithm: Needleman and Wunsch, J Mol. Biol. 48:443-453 (1970); Comparison matrix: BLOSSUM62 from Hentikoff and Hentikoff, Proc. Natl. Acad. Sci. USA 89:10915-10919 (1992); Gap Penalty: 12; Gap Length Penalty: 4. A program that can be used with these parameters is publicly available as the “gap” program from Genetics Computer Group (“GCG”), Madison, Wis. The above parameters along with no penalty for end gap are the default parameters for peptide comparisons. In one embodiment the BLASTP program of NCBI is used with the default parameters of no compositional adjustment, expect value of 10, word size of 3, BLOSUM62 matrix, gap extension cost of 11, end gap extension cost of 1, dropoff (X) for blast extension (in bits) 7, X dropoff value for gapped alignment (in bits) 15, and final X dropoff value for gapped alignment (in bits) 25.

Other sequences encompassed include those containing conserved amino acid substitutions. A “conservative amino acid substitution” is one in which the amino acid residue is replaced with an amino acid residue having a similar side chain, or physicochemical characteristics (e.g., electrostatic, hydrogen bonding, isosteric, hydrophobic features). Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, methionine, cysteine), nonpolar side chains (e.g., alanine, valine, leucine, isoleucine, proline, phenylalanine, tryptophan), β-branched side chains (e.g., threonine, valine, isoleucine) and aromatic side chains (e.g., tyrosine, phenylalanine, tryptophan, histidine).

Another embodiment of the present application comprises polynucleotides that encode the novel nmGLP-1 peptides analogs herein. These polynucleotides include both DNA and RNA sequences. Due to the degeneracy of the genetic code, the polynucleotides can include mutations that are “silent”, i.e., mutated and non-mutated codons encode the same amino acid, or mutations that result in conservative amino acids substitutions.

nmGLP-1 peptides or analogs thereof are useful in view of their pharmacological properties. In particular, nmGLP-1 peptides or analogs thereof possess activity as agents to control appetite, reduce food intake, and both control appetite and reduce food intake in a mammal, for example a human. They can be used to treat conditions or diseases which can be alleviated by preventing or reducing obesity, reducing body weight, controlling appetite, and/or reducing food intake in a mammalian subject, for example a human. In one embodiment, the subject further suffers from impaired glucose tolerance, insulin resistance or diabetes mellitus. In other embodiments the nmGLP-1 peptides, and in particular SEQ ID NO; 1, 2, and 6-56, are used for the treatment of insulin resistance, impaired glucose tolerance, diabetes mellitus, including Type II diabetes and gestational diabetes, or other disorders which would be benefited by agents that stimulate insulin secretion. The peptides and pharmaceutical compositions of the present invention can also be used to treat irritable bowel syndrome (see WO 99/64060), or to reduce the morbidity and mortality associated with myocardial infarction (see WO 93/08531) and stroke (see WO 00/16797) as well as catabolic changes that occur after surgery (see U.S. Pat. No. 6,006,753), or related conditions in mammals such as humans, and to reduce the mortality and morbidity that occurs in critically ill patients that experience respiratory distress or have illness or condition that is likely to lead to respiratory distress. Examples of conditions that involve respiratory distress include acute lung injury, respiratory distress syndrome, cor pulmonale, chronic obstructive pulmonary disease, and sepsis. Also included are subjects at risk for developing non-insulin dependent diabetes (see WO 00/07617), such as those with prediabetes. In one aspect, the nmGLP-1 peptides or analogs thereof do not include natural non-mammalian GLP-1 peptides. In one particular aspect the nmGLP-1 peptides do not include SEQ ID NO 3-5. In another aspect, the nmGLP-1 peptides further do not include any one of, any subgroup of, or all of SEQ ID NOS: 1, 2 and 48-52.

nmGLP-1 peptides or analogs thereof can form salts with various inorganic and organic acids and bases. Such salts include salts prepared with organic and inorganic acids, for example, HCl, HBr, H₂SO₄, H₃PO₄, trifluoroacetic acid, acetic acid, formic acid, methanesulfonic acid, toluenesulfonic acid, maleic acid, fumaric acid and camphorsulfonic acid. Salts prepared with bases include ammonium salts, alkali metal salts, e.g., sodium and potassium salts, alkali earth salts, e.g., calcium and magnesium salts, and zinc salts. The salts can be formed by conventional means, as by reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

nmGLP-1 peptides or analogs thereof can also be formulated as pharmaceutically acceptable salts (e.g., acid addition salts) and/or complexes thereof. Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the therapeutic agent.

Pharmaceutically acceptable salts include acid addition salts such as those containing sulfate, hydrochloride, phosphate, sulfamate, acetate, citrate, lactate, tartrate, succinate, oxalate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts can be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

nmGLP-1 peptides or analogs thereof can be formulated as pharmaceutical compositions for use in conjunction with the methods of the present application. Compositions useful in the application can conveniently be provided in the form of formulations suitable for parenteral (including intravenous, intramuscular and subcutaneous) or nasal or oral administration. In some cases, it will be convenient to provide an nmGLP-1 peptide or analog thereof and another appetite-suppressing, food-intake-reducing, plasma glucose-lowering or plasma lipid-lowering agent, such as amylin, an amylin agonist, a CCK, or a leptin, in a single composition or solution for administration together. In other cases, it can be more advantageous to administer the additional agent separately from the nmGLP-1 peptide or analog thereof. A suitable administration format can best be determined by a medical practitioner.

Compounds can be provided as parenteral compositions for injection or infusion. They can, for example, be suspended in inert oil, for example, a vegetable oil such as sesame, peanut, olive oil, or other acceptable carrier. In one embodiment, they are suspended in an aqueous carrier, for example, in an isotonic buffer solution at a pH of about 3.0 to about 8.0, at a pH of about 3.0 to about 7.0, at a pH of about 3.0 to about 6.0, at a pH of about 3.5 to about 6.0, or at a pH of about 3.5 to about 5.0. In alternative embodiments, the pH can be adjusted to a pH range from about 5.0 to about 8.0. These compositions can be sterilized by conventional sterilization techniques, or can be sterile filtered. The compositions can contain pharmaceutically acceptable auxiliary substances as required to approximate physiological conditions, such as pH buffering agents. Exemplary buffers include for example, sodium acetate/acetic acid buffer, although any buffer system that provides adequate buffering capacity in the desired pH range can be used. A form of repository or “depot” slow release preparation can be used so that therapeutically effective amounts of the preparation are delivered into the bloodstream over many hours, days or weeks following transdermal injection or delivery. Numerous examples of depot or nasal methods of administration are known in the art.

In one aspect, methods for reducing or preventing obesity, reducing body weight, controlling or reducing food intake, controlling or reducing appetite, reducing spontaneous food intake are provided wherein the method comprises chronically administering an effective amount of an nmGLP-1 or analog thereof to a subject in need or desirous thereof. In one aspect, the nmGLP-1 or analog thereof can be administered in an extended or slow release formulation. In one embodiment, the nmGLP-1 or analog thereof can be administered in a polymer-based sustained release device. Such polymer-based sustained release devices are described, for example, in U.S. patent application Ser. No. 11/107,550, filed Apr. 15, 2005, and U.S. patent application Ser. No. 11/104,877 (U.S. Patent Publication 2005/0271702), filed Apr. 13, 2005 which are incorporated herein by reference in their entireties.

If desired, isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is particularly useful for buffers containing sodium ions.

Suitable excipients can be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, cyclodextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients. Other examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents.

Certain nmGLP-1 peptides or analogs thereof may be substantially insoluble in water and sparingly soluble in most pharmaceutically acceptable protic solvents and in vegetable oils. However, the compounds can be soluble in medium chain fatty acids (e.g., caprylic and capric acids) or triglycerides and have high solubility in propylene glycol esters of medium chain fatty acids. Also included are compositions, which have been modified by substitutions or additions of chemical or biochemical moieties which make them more suitable for delivery (e.g., increase solubility, bioactivity, palatability, decrease adverse reactions, etc.), for example by esterification, glycation, PEGylation, etc.

An nmGLP-1 peptide or analog thereof can also be formulated for oral administration in a self-emulsifying drug delivery system (SEDDS). Lipid-based formulations such as SEDDS are particularly suitable for low solubility compounds, and can generally enhance the oral bioavailability of such compounds. Cyclodextrins can be added as aqueous solubility enhancers. Cyclodextrins include methyl, dimethyl, hydroxypropyl, hydroxyethyl, glucosyl, maltosyl and maltotriosyl derivatives of α-, β-, and γ-cyclodextrin. An exemplary cyclodextrin solubility enhancer is hydroxypropyl-β-cyclodextrin (HPBC), which can be added to any of the above-described compositions to further improve the aqueous solubility characteristics of an nmGLP-1 peptide, agonist, or analog thereof. In one embodiment, the composition comprises 0.1% to 20% hydroxypropyl-β-cyclodextrin, 1% to 15% hydroxypropyl-β-cyclodextrin, or from 2.5% to 10% hydroxypropyl-β-cyclodextrin. The amount of solubility enhancer employed will depend on the amount of the nmGLP-1 peptide, agonist, or analog thereof in the composition.

Absorption enhancers can be added including, but not limited to, cationic polyamino acids, such as poly-arginine, poly-histidine and poly-lysine. Other suitable absorption enhancing agents include chitosan and phospholipids such as didecanoyl phosphatidylcholine (DDPC).

If desired, solutions of the compositions described herein can be thickened with a thickening agent such as methyl cellulose. They can be prepared in emulsified form, either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton).

Compositions can be prepared by mixing the ingredients following generally accepted procedures. For example, the selected components can be simply mixed in a blender or other standard device to produce a concentrated mixture which can then be adjusted to the final concentration and viscosity by the addition of water or thickening agent and possibly a buffer to control pH or an additional solute to control tonicity.

An optimal formulation and mode of administration of compounds of the present application to a subject depend on factors known in the art such as the particular disease or disorder, the desired effect, and the type of subject. While the compounds will typically be used to treat human subjects they can also be used to treat similar or identical diseases, conditions or disorders in other mammals such as other primates, farm animals such as swine, sheep, goats and cattle, and sports animals and pets such as horses, dogs and cats.

According to the methods disclosed herein, an nmGLP-1 peptide or analog thereof can be: (1) co-formulated and administered or delivered simultaneously in a combined formulation; (2) delivered by alternation or in parallel as separate formulations; or (3) by any other combination therapy regimen known in the art. When delivered in alternation therapy, the methods can comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy can also be used.

Compositions comprise a therapeutically-effective amount of at least one nmGLP-1 peptide or analog thereof and a pharmaceutically acceptable carrier or excipient. Such carriers include, but are not limited to, saline, buffered saline, dextrose, water, glycerol, ethanol, lactose, phosphate, mannitol, arginine, trehalose and combinations thereof. The formulations can be formulated to suit the mode of administration. Although not limited to the following ranges and provided as an illustration, suggested dose ranges for various applications are 0.1 to 5.0×10⁻¹² mol/kg min for intravenous administration; 0.1 to 5.0 nmol/kg nmGLP-1 (naturally-occurring nmGLP-1 and/or at least one of its analogs or agonists) in any form as a single subcutaneous injection; continuous subcutaneous administration in a range from about 0.2 to 20×10⁻¹² mol/kg min. It is believed that the subcutaneous amounts can be up to or at least 10 times higher than those for intravenous application.

An effective daily dose of an nmGLP-1 peptide, agonist, or analog, can be in the range of about 10 μg to about 5 mg/day, about 10 μg to about 2 mg/day and about 10 μg to 100 μg to about 1 mg/day, or about 30 μg to about 500 μg/day, for an exemplary 70 kg patient, administered in a single or divided doses. It should be noted that a 70 kg patient is for exemplary purposes only and that doses can be calculated on a per kg basis using a 70 kg patient. The exact dose to be administered is determined by the attending clinician and is dependent upon where the particular compound lies within the above quoted range, as well as upon the age, weight and condition of the individual. Administration typically begins whenever the reduction, prevention, treatment or alleviation of the symptom or affliction, for example, suppression of food intake, appetite control, weight lowering or normalization of glycemia is desired, for example, at the first sign of symptoms or shortly after diagnosis of obesity.

For any nmGLP-1 peptide, agonist, or analog thereof the effective amount can be estimated initially either in cell culture assays, e.g., in animal models, such as rat or mouse models. An animal model can also be used to determine the appropriate concentration range and route of administration. Such information can then be used to determine useful doses and routes for administration in humans.

Efficacy and toxicity can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The data obtained from cell culture assays and animal studies can be used in formulating a range of dosage for human use. The dosage contained in such compositions is preferably within a range of circulating concentrations that include an ED₅₀ with little or no toxicity. The dosage can vary within this range depending upon the dosage form employed, sensitivity of the patient, and the route of administration.

In accordance with the methods and uses of the present application, nmGLP-1 peptides or analogs thereof, can be administered in any manner known in the art that renders such molecules biologically available to the subject or sample in an effective amount. For example, nmGLP-1 peptides or analogs thereof, can be administered to a subject via any central or peripheral route known in the art including, but not limited to: oral, parenteral, transdermal, transmucosal, or pulmonary routes.

In one embodiment, administration is parenteral. In another embodiment, the mode of administration is peripheral (subcutaneous or intravenous). A particular route of administration is subcutaneous. In other aspects, the peripheral administration is selected from the group consisting of buccal, nasal, pulmonary, oral, intraocular, rectal, and transdermal administration. Further, nmGLP-1 peptides or analogs thereof, can be administered to a sample via pouring, pipetting, immersing, injecting, infusing, perfusing, or any other means known in the art. Determination of the appropriate administration method is usually made upon consideration of the condition (e.g., disease or disorder) to be treated, the stage of the condition (e.g., disease or disorder), the comfort of the subject, and other factors known to those of skill in the art.

Administration by methods disclosed herein or known in the art can be intermittent or continuous, both on an acute and/or chronic basis. Acute administration includes a temporary administration for a period of time before, during and/or after the occurrence of a transient event. An acute administration generally entails an administration that is indicated by a transient event or condition.

Chronic or continuous administration can be warranted where no particular transient event or transient condition is identified. Chronic or continuous administration includes administration of an nmGLP-1 peptide or analog thereof for an indefinite period of time on the basis of a general predisposition a non-transient condition. When a GLP-1 peptide or analog thereof is administered chronically or continuously, administration can continue for any length of time. However, chronic or continuous administration often occurs for an extended period of time. For example, in one embodiment, chronic or continuous administration continues for 4, 6, 8, 24, 48 or 72 hours. In another embodiment, chronic or continuous administration continues for 96 hours, 120 hours, 144 hours, 1 week, 2 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, 9 months, 1 year, 2 years or longer. Although time periods may overlap, chronic and continuous administration are not synonymous. Chronic administration contemplates delivery of the pharmaceutical composition for extended periods of time either by regular discrete delivery method, for example injection or taking a pill, or continuous infusion. Continuous administration contemplates continuous delivery of the pharmaceutical composition as in, for example, continuous intravenous or subcutaneous infusion.

An embodiment comprises a composition, in particular a pharmaceutical composition, containing at least one naturally-occurring nmGLP-1 peptide in combination with a pharmaceutically-acceptable excipient. This composition can be administered to mammals and especially humans for a variety of reasons as discussed herein, including appetite suppression, controlling weight loss, normalization of blood glucose, and reducing the urge to intake food. Another embodiment of the present application comprises a composition containing at least one nmGLP-1 or nmGLP-1 analog in combination with a pharmaceutically-acceptable excipient. These nmGLP-1 or nmGLP-1 analogs can also be used in combination with a suitable pharmaceutical carrier for any of the uses or treatment methods described herein, including appetite control and/or reduction of food intake.

In one embodiment, the method includes administration of nmGLP-1 peptides or analogs thereof to reduce obesity. The reduction in obesity can be measured by any method known in the art. As used herein, reduction in obesity is measured as a reduction in a subject's body mass index (BMI). In an example, a reduction in obesity after treatment with an nmGLP-1 peptide or agonist or analog thereof can be a reduction of 1, 2, 3, 4, 5, 6, 7, 8, 9, 10, 11, 12, 13, 14, 15 or more BMI points of the subject's pre-treatment BMI. In another embodiment, treatment using the methods and compositions disclosed herein result in a decrease of BMI to less than 30. In another embodiment, BMI is reduced to less than 25. In still another embodiment, BMI is reduced to between 18.5 and 24.9. As used herein, a subject's body mass index can be calculated as the subject's weight in kilograms divided by the square of the subject's height in meters.

In another embodiment, an nmGLP-1 peptide or analog thereof can be used for preventing or treating obesity in combination with other obesity treating or reducing compounds. Examples of obesity treating or reducing compounds include amylin, an amylin agonist analog, a CCK or CCK agonist, or a leptin or leptin agonist, or an exendin or exendin agonist analog, or a PYY or a PYY analog. Such combinations can be combined in a single composition or solution for administration together. In other cases, it may be more advantageous to administer the additional agent separately from the nmGLP-1 peptide or analog thereof.

Another aspect includes methods for reducing body weight of a subject comprising administering to a subject an effective amount of an GLP-1 peptide or analog thereof to a subject desirous or in need thereof. In one embodiment, the methods of the invention are used to reduce the body weight of an obese subject, an overweight subject or a subject desirous of reducing body weight. Reduction in a subject's body weight can be measured using any method available. As used herein, the reduction in body weight is measured as a reduction in a subject's weight in pounds or kilograms. In one embodiment, a subject's body weight is reduced 5, 10, 15, 20, 25, 30, 35, 40, 45, 50, 55, 60, 65, 70, 75, 80, 85, 90, 95, 100, 110, 120, 130, 140, 150 or more pounds from the subject's pre-treatment body weight.

In another embodiment, a reduction in a subject's body weight can be measured as a percent decrease in the subject's weight compared to the subject's weight prior to treatment. For example, a subject's body weight can be reduced 2% 5%, 7%, 10%, 12%, 15%, 17%, 20%, 22%, or 25% or more from the subject's pre-treatment body weight.

Reduction in body weight or obesity can be measured over any suitable period, for example, weekly, monthly, three monthly, six-monthly or at a certain periodicity, such as 7, 10, 15, 20, 25, 30 days over a period of time such as a month, three months, six months, or a year or more period.

In another embodiment, an nmGLP-1 peptide or analog thereof can be used for reducing body weight in combination with other body weight reducing compounds. Examples of body weight reducing compounds include amylin, an amylin agonist analog, a CCK or CCK agonist, or a leptin or leptin agonist, or an exendin or exendin agonist analog, or a PYY or a PYY analog. Such combinations can be combined in a single composition or solution for administration together. In other cases, it may be more advantageous to administer the additional agent separately from an nmGLP-1 peptide or analog thereof.

Another aspect includes methods for reducing food intake of a subject comprising administering to a subject an effective amount of an nmGLP-1 peptide or analog thereof, such as those disclosed herein to a subject desirous or in need thereof. In a one embodiment, the methods of the invention are used to reduce the food intake of an obese subject, including a subject a subject desirous of reducing food intake. Reduction in a subject's food intake can be measured using any method available. In one embodiment, the reduction in food intake can be measured as a reduction in a subjects caloric ingestion per day. In an example, a subject's average daily caloric ingestion is reduced 50, 75, 100, 150, 200, 250, 300, 350, 400, 450, 500, 550, 650, 700, 750, 800, 850, 900, 950, 1000 or more Calories from the subject's pre-treatment average caloric ingestion per day. As used herein, Calories refers to a nutritional calorie also known as a large calorie or kilogram calorie. A nutritional calorie is a unit expressing a heat-producing or energy-producing value in food that when oxidized in the body is capable of releasing one large calorie of energy (1000 gram calories or 3.968 Btu).

In another embodiment, an nmGLP-1 peptide or analog thereof, such as those described herein, can be used for reducing food intake in combination with other food intake reducing compounds. Examples of food intake reducing compounds include amylin, an amylin agonist analog, a CCK or CCK agonist, or a leptin or leptin agonist, or an exendin or exendin agonist analog, or a PYY or a PYY analog. Such combinations can be combined in a single composition or solution for administration together. In other cases, it may be more advantageous to administer the additional agent separately from the nmGLP-1 peptide or analog thereof.

In one embodiment, the methods are used to treat or prevent conditions or disorders which can be alleviated by reducing nutrient availability in a subject in need thereof, comprising administering to said subject a therapeutically or prophylactically effective amount of a of an nmGLP-1 or nmGLP-1 analog, such as those disclosed herein. Such conditions and disorders include, but are not limited to, hypertension, dyslipidemia, cardiovascular disease, eating disorders, insulin-resistance, obesity, and diabetes mellitus of any kind.

One aspect provides an nmGLP-1 peptide or analog thereof that has greater stability as compared to a mammalian GLP-1 peptide. Another aspect provides a method for increasing the stability of a peptide, especially to enzymatic degradation such as takes place in blood or serum or plasma. In one embodiment, the peptide is an insulinotropic peptide, while in another embodiment, the peptide is an incretin or incretin mimetic, for example a GLP-1 or an exendin. In one embodiment, the degradation is due to the action of a dipeptidyl peptidase, for example dipeptidyl peptidase IV (DPPIV). In one embodiment, the peptide having increased stability comprises a C-terminal amino acid sequence comprising the amino acid sequence C terminal to position 28 of an nmGLP-1 peptide or any one of the peptides of Table 2. In another embodiment, the peptide having increased stability consists essentially of a C-terminal amino acid sequence comprising the amino acid sequence C terminal to position 28 of an nmGLP-1 peptide or any one of the peptides of Table 2. In still another embodiment, the peptide having increased stability comprises a C-terminal amino acid sequence consisting of the amino acid sequence C terminal to position 28 of an nmGLP-1 peptide or any one of the peptides of Table 2. Amino acid sequences that can be used to increase peptide stability, include, but are not limited to any one of the amino acid sequence found in Table 2. In one embodiment, the amino acid sequence used to increase stability comprises at least 5 consecutive amino acids of any of the sequences in Table 2. In another embodiment, the amino acid sequence used to increase stability comprises at least the first 5 amino acids of any one of the sequences in Table 2. In one embodiment, the amino acid sequence may be added to the C-terminus of a peptide in order to increase stability. In another embodiment, the stability increasing amino acid sequence may be substituted on a one-for-basis with the consecutive C-terminal amino acids in the peptide whose stability is to be increased. For example, if the amino acid sequence used to increase stability is 10 amino acids long, it would replace the 10 C-terminal amino acids in the peptide whose stability is to be increased. In yet another embodiment, the stability increasing amino acid sequence is added using a combination of the preceding methods such that only some of the C-terminal amino acids of the peptide whose stability is to be increased are substituted with the remain amino acids of the stability increasing sequence comprising new amino acids, thereby increasing the length of the peptide. Similar additions can be made to the N-terminus. Peptides comprising the stability increasing amino acid sequences described herein can be made by any method know in the art, for example, any of the peptide synthesis or recombinant methods disclosed herein.

Accordingly, in one embodiment the stabilized peptides are exendin analogs having amino acids 1-27, 1-28, 1-29 or 1-30 of the exendins taught in U.S. Pat. No. 6,767,887 with the addition of a stabilizing C-terminal amino acid sequence as taught herein, obtained from an nmGLP-1 peptide or Table 2. In another embodiment the stabilized peptides are exendin analogs having amino acids 1-27, 1-28, 1-29, 1-30, 1-31, 1-32, 1-33, or 1-34 of the exendins taught in U.S. Pat. No. 7,226,990 with the addition of a stabilizing C-terminal amino acid sequence as taught herein, obtained from an nmGLP-1 peptide or Table 2. In yet a further embodiment, the stabilized peptides are GLP-1 analogs having amino acids 1-27, 1-28, 1-29, 1-30, 1-31, 1-32, 1-33, or 1-34 of the GLP-1 peptides, including fatty acyl derivatives, e.g. liraglutide, taught in U.S. Pat. No. 7,226,990 with the addition of a stabilizing C-terminal amino acid sequence as taught herein, obtained from an nmGLP-1 peptide or Table 2.

In one embodiment, a peptide is considered to have increased stability if it shows resistance to degradation greater than the native or naturally-occurring peptide upon which it is based. In one embodiment stability is determined by the percent of peptide which remains undegraded (i.e. intact) following exposure to a peptidase of interest for a set period of time. For example, in one embodiment, a peptide is considered to have increase stability if at least 90% of the peptide is intact, i.e. resists degradation, after incubation in a brush border membrane (BBM) assay such as described herein, for 5 hours at 37° C. In another embodiment, a peptide is considered to have increase stability if at least 75% of the peptide is intact, i.e. resists degradation, after incubation in BBM assay, for 5 hours at 37° C. In another embodiment, the peptide is considered to have increase stability if at least 90% of the peptide is intact, i.e. resists degradation, after incubation in the presence of DDPIV such as described herein, for 1 hour at 37° C. In still another embodiment, the peptide is considered to have increase stability if at least 75% of the peptide is intact, i.e. resists degradation, after incubation in the presence of DDPIV, for 1 hour at 37° C.

TABLE 2

| SEQ ID NO | Sequence |
|---|---|
| 57 | GGPSKEIIS |
| 58 | GKPKKIRYS |
| 59 | GGPSSGAPPPS |
| 60 | PGSEGIKSI |
| 61 | GQPKPDAVET |
| 62 | IDWLIKGQ |
| 63 | IDWLIKGQG |
| 64 | GQPKPDAVETRSVDTL |
| 65 | GKPKKQRLS |
| 66 | YADAPYIS |
| 67 | VGGGSTMEDDTEPLS |
| 68 | GQPKQDVGTNRGTNL |

In a further embodiment of each one of the embodiments herein where receptor agonism is desired, excluded are peptides SEQ ID NO: 29, 30, 37, 39, 46, and 47. For example, an nmGLP-1 peptide, or any of the methods using a nmGLP-1 peptide, would comprise or use a polypeptide selected from at least one of the group consisting of SEQ ID NO: 6 to SEQ ID NO: 47 and SEQ ID NO: 53 to SEQ ID NO: 56 and excluding SEQ ID NO: 29, 30, 37, 39, 46, and 47. In another embodiment the nmGLP-1 peptide comprises those from Table 2 that have an EC50 of less than 100, less than 50, less than 10, less than 1 and less than 0.5 nM for the GLP-1 cAMP assays described herein and excludes the known GLP-1 peptides and exendins.

The following examples are intended to provide illustrations of the application of the present invention. The following examples are not intended to completely define or otherwise limit the scope of the invention.

### EXAMPLES

### Example 1

### Synthesis of Polypeptides

GLP-1 peptides can be synthesized using standard polypeptide synthesis methods. Such methods are described below and in U.S. Pat. No. 6,610,824 and U.S. Pat. No. 5,686,411 and in patent application Ser. No. 454,533 (filed Dec. 6, 1999), the entireties of which are incorporated herein by reference.

Polypeptides were synthesized on a Pioneer continuous flow peptide synthesizer (Applied Biosystems) using PAL-PEG-PS resin (Applied Biosystems) with a loading of 0.2 mmol/g (0.25 mmole scale). Fmoc amino acid (4.0 eq, 1.0 mmol) residues were activated using 4.0 eq HBTU, 4.0 eq of HOBT, 8.0 eq DIEA and coupled to the resin for 1 hour. The Fmoc group was removed by treatment with 20% (v/v) piperidine in dimethylformamide. Final deprotection and cleavage of the peptide from the solid support was performed by treatment of the resin with reagent B (93% TFA, 3% phenol, 3% water and 1% triisopropylsilane) for 2-3 hours. The cleaved peptide was precipitated using tert-butyl methyl ether, pelleted by centrifugation and lyophilized. The pellet was re-dissolved in water (10-15 mL), filtered and purified via reverse phase HPLC using a C-18 column and an acetonitrile/water gradient containing 0.1% TFA. The purified product was lyophilized and analyzed by ESI-LC/MS and analytical HPLC and were demonstrated to be pure (>98%). Mass results all agreed with calculated values.

Alternatively, peptides were assembled on a Symphony® peptide synthesizer (Protein Technologies, Inc., Woburn, Mass.) using Rink amide resin (Novabiochem, San Diego, Calif.) with a loading of 0.43-0.49 mmol/g at 0.050-0.100 mmol. Fmoc amino acid (Applied Biosystems, Inc. 5.0 eq, 0.250-0.500 mmol) residues were dissolved at a concentration of 0.10 M in 1-methyl-2-pyrrolidinone. All other reagents (HBTU, 1-hydroxybenzotriazole hydrate and N,N-diisopropylethylamine) were prepared as 0.55 M dimethylformamide solutions. The Fmoc protected amino acids were then coupled to the resin-bound amino acid using, HBTU (2.0 eq, 0.100-0.200 mmol), 1-hydroxybenzotriazole hydrate (1.8 eq, 0.090-0.18 mmol), N,N-diisopropylethylamine (2.4 eq, 0.120-0.240 mmol) for 2 hours. Following the last amino acid coupling, the peptide was deprotected using 20% (v/v) piperidine in dimethylformamide for 1 hour. Once the peptide sequence is completed, the Symphony® peptide synthesizer is programmed to cleave the resin. Trifluoroacetic acid (TFA) cleavage of the peptide from resin was carried out using a reagent mixture composed of 93% TFA, 3% phenol, 3% water and 1% triisopropylsilane. The cleaved peptide was precipitated using tert-butyl methyl ether, pelleted by centifugation and lyophilized. The pellet was dissolved in acetic acid, lyophilized and then dissolved in water, filtered and purified via reverse phase HPLC using a C18 column and an acetonitrile/water gradient containing 0.1% TFA. Anaytical HPLC was used to assess purity of peptide and identity was confirmed by LC/MS and MALDI-MS.

### Example 2

### GLP-1 Receptor Binding Assay

GLP-1 receptor binding activity and affinity can be measured using a binding displacement assay in which the receptor source is RINm5F cell membranes, and the ligand is [¹²⁵I]GLP-1. Homogenized RINm5F cell membranes are incubated in 20 mM HEPES buffer with 40,000 cpm [¹²⁵I]GLP-1 tracer, and varying concentrations of test compound for 60 minutes at 23° C. with constant mixing. Reaction mixtures are filtered through glass filter pads presoaked with 0.3% PEI solution and rinsed with ice-cold phosphate buffered saline. Bound counts are determined using a scintillation counter. Binding affinities are calculated using GraphPad Prism software (GraphPad Software, Inc., San Diego, Calif.).

In addition, receptor binding can be measured using cells prepared from confluent cultures of RINm5f cells expressing endogenous GLP-1, calcitonin, and GIP receptors. Cells, and peptide or buffer solution are combined in 384-well plates. After a 30-minute incubation at room temperature in air, cAMP content is measured as recommended in the Perkin-Elmer AlphaScreen kit instructions.

The results of these binding assays are provided in Table 3 below.

TABLE 3

| | | GLP/GIP/CT | GLP-1 |
|---|---|---|---|
| | GLP RBA IC50 | cAMP(RIN) EC50 | cAMP (6-23) |
| SEQ ID NO | nM | nM | EC50 nM |
| 1 | 3 | 1.9 | 7.7 |
| 2 | 0.1 | 0.36 | 0.82 |
| 3 | 0.148 | 0.285 | 0.14 |
| 4 | 0.517 | 0.23 | 0.035 |
| 5 | 0.58 | Nd | 0.82 |
| 6 | 1 | 1.23 | nd |
| 7 | 3 | 4.7 | nd |
| 8 | 8.1 | 19 | nd |
| 9 | 4.2 | 0.28 | nd |
| 10 | 1.9 | 0.048 | nd |
| 11 | 2.3 | 0.59 | nd |
| 12 | 8.8 | 0.29 | nd |
| 13 | 74 | 196 | nd |
| 14 | 18 | 18 | 2.2 |
| 15 | 2.6 | Nd | 1.15 |
| 16 | 60 | Nd | 9.5 |
| 17 | 46 | Nd | 5.5 |
| 18 | 40 | Nd | 6.8 |
| 19 | 0.62 | 0.11 | nd |
| 20 | 0.47 | 0.11 | nd |
| 21 | 2.1 | 1 | nd |
| 22 | 0.067 | 0.1 | nd |
| 23 | 0.074 | 0.22 | nd |
| 24 | 1.3 | 0.85 | nd |
| 25 | 0.54 | 0.53 | nd |
| 26 | 0.11 | 0.967 | 0.16 |
| 27 | 0.1 | 0.544 | 0.11 |
| 28 | 0.067 | Nd | 0.079 |
| 29 | 91 | Nd | 930 |
| 30 | 537 | Nd | >1000 |
| 31 | 19 | Nd | 44 |
| 32 | 22 | Nd | 8.3 |
| 33 | 81 | Nd | 145 |
| 34 | 15 | Nd | 0.095 |
| 35 | 0.28 | Nd | 0.172 |
| 36 | 0.36 | Nd | 0.086 |
| 37 | 22 | Nd | >1000 |
| 38 | 42 | Nd | 104 |
| 39 | 61 | Nd | >1000 |
| 40 | 0.62 | Nd | 0.12 |
| 41 | 9 | Nd | 220 |
| 42 | Nd | Nd | 0.22 |
| 43 | Nd | Nd | 0.14 |
| 44 | Nd | Nd | 23 |
| 45 | Nd | Nd | 34 |
| 46 | Nd | Nd | 1945 |
| 47 | Nd | Nd | 2846 |
| 48 | 34 | 14 | 2.344 |
| 49 | 74 | 415 | nd |
| 50 | 0.06 | 0.14 | 0.082 |
| 51 | 110.7 | 971 | nd |
| 52 | 0.9 | 382 | nd |
| 53 | 0.052 | 0.146 | 0.12 |
| 54 | 0.067 | 0.435 | 0.14 |
| 55 | 0.05 | 0.576 | 0.14 |
| 56 | 0.092 | Nd | 0.136 |

nd = not done

### Example 3

### Supplementary GLP-1 Cyclase Assay

GLP-1 cyclase activity can be measured using an assay in which the cells are prepared from confluent cultures of rat thyroid carcinoma 6-23 clone 6 cells or RINm5F insulinoma cells expressing endogenous GLP-1 receptors. Cells, and peptide or buffer solutions are combined in 384-well plates and incubated at room temperature in air for 30-minutes. cAMP content can then be measured according to recommendations in the Perkin-Elmer AlphaScreen™ kit instructions using the Perkin Elmer Fusion fluorimeter and data are analyzed using XLfit software (IDBS).

### Example 4

### Effects on Caloric Intake (FI Assay)

The effect of various test peptides on food intake is investigated using an acute food intake assay. This assay measures food consumption in lean, group-housed, overnight-fasted NIH/Swiss mice. Twelve mice are housed three to a cage. A polypeptide is injected intra-peritoneally in mice and food intake is measured during the next two hours. Decreased, as well as increased, food intake is measured. Testing of the various doses of the polypeptide generates ED50s.

Female NIH/Swiss mice (8-24 weeks old) are group housed with a 12:12 hour light:dark cycle with lights on at 0600. Water and a standard pelleted mouse chow diet are available ad libitum, except as noted. Animals are fasted starting at approximately 1500 hrs, 1 day prior to experiment. The morning of the experiment, animals are divided into experimental groups. In a typical study, n=4 cages with 3 mice/cage.

At time=0 min, all animals are given an intraperitoneal injection of vehicle or compound in an amount ranging from about 63 to 1000 μg/kg, and immediately given a pre-weighed amount (10-15 g) of the standard chow. Food is removed and weighed at 30, 60, 120 and 180 min to determine the amount of food consumed (Morley, Flood et al., Am. J. Physiol. 267: R178-R184, 1994). Food intake is calculated by subtracting the weight of the food remaining after the e.g., 30, 60, 120, and 180 minute time point from the weight of the food provided initially at time=0 and data are expressed as % inhibition of food intake, relative to vehicle controls.

The results of the food intake assay are provided in Table 4 below.

TABLE 4

| | | |
|---|---|---|
| | Dose | Food Intake |
| SEQ | Tested | % inhibition |

| ID NO: | μg/kg | 30 min | 60 min | 120 min | 180 min |
|---|---|---|---|---|---|
| 1 | 1000 | 56 | 69 | 73 | 72 |
| 2 | 1000 | 41 | 53 | 61 | 55 |
| 3 | 1000 | 13 | 13 | 11 | ND |
| 4 | 75 | 49 | 63 | 68 | 67 |
| 5 | 63 | 50 | 62 | 49 | 49 |
| 6 | 63 | 50 | 62 | 49 | 49 |
| 7 | 1000 | 28 | 28 | 22 | 19 |
| 8 | 1000 | 37 | 43 | 42 | 35 |
| 9 | 1000 | 33 | 51 | 62 | 57 |
| 10 | 1000 | 49 | 59 | 69 | 68 |
| 11 | 1000 | 48 | 65 | 69 | 68 |
| 12 | 1000 | 40 | 57 | 57 | 45 |
| 13 | 1000 | 33 | 38 | 16 | 5 |
| 14 | 1000 | 33 | 45 | 31 | 18 |
| 15 | 1000 | 70 | 81 | 83 | 84 |
| 1:6 | 1000 | 63 | 76 | 79 | 81 |
| 17 | 1000 | 49 | 68 | 77 | 79 |
| 18 | 1000 | 14 | 40 | 59 | 62 |
| 19 | 1000 | 51 | 67 | 75 | 77 |
| 20 | 1000 | 43 | 55 | 70 | 71 |
| 21 | 1000 | 48 | 64 | 73 | 76 |
| 22 | 1000 | 54 | 66 | 64 | 63 |
| 23 | 1000 | 47 | 66 | 73 | 75 |
| 24 | 1000 | 48 | 53 | 45 | 42 |
| 25 | 1000 | 58 | 59 | 54 | 32 |
| 26 | 1000 | 55 | 68 | 75 | 75 |
| 27 | 1000 | 45 | 60 | 67 | 70 |
| 28 | 1000 | 44 | 60 | 69 | 62 |
| 29 | 1000 | −2 | 2 | 1 | ND |
| 30 | 1000 | 8 | 7 | 3 | ND |
| 31 | 1000 | −2 | 3 | 4 | ND |
| 32 | 1000 | 13 | 19 | 10 | ND |
| 33 | 1000 | 18 | 18 | 5 | ND |
| 34 | 1000 | 28 | 45 | 54 | ND |
| 35 | 1000 | 33 | 38 | 36 | ND |
| 36 | 1000 | 36 | 52 | 60 | ND |
| 37 | 1000 | −3 | −3 | −7 | ND |
| 38 | 1000 | 9 | 2 | −2 | 0 |
| 39 | 1000 | −13 | 0 | 5 | 4 |
| 40 | 1000 | 34 | 45 | 45 | 37 |
| 41 | 1000 | 1 | −1 | 9 | −1 |
| 42 | 1000 | 55 | 64 | 54 | 33 |
| 43 | 1000 | 52 | 69 | 77 | 77 |
| 44 | 1000 | 17 | 16 | 9 | −6 |
| 45 | 1000 | 19 | 30 | 22 | 4 |
| 46 | 1000 | −5 | −5 | −3 | −4 |
| 47 | 1000 | 5 | −8 | −5 | −27 |
| 48 | 1000 | 48 | 40 | 8 | ND |
| 49 | 1000 | 40 | 34 | 14 | ND |
| 50 | 1000 | 41 | 51 | 50 | ND |
| 51 | 1000 | −6 | −3 | 9 | ND |
| 52 | 1000 | 18 | 14 | 6 | ND |
| 53 | 1000 | 40 | 44 | 50 | 34 |
| 54 | 1000 | 45 | 56 | 62 | 60 |
| 55 | 1000 | 52 | 64 | 69 | 72 |
| 56 | 1000 | 50 | 66 | 71 | 72 |

### Example 5

### Assessment of Stability by the Kidney Brush Border Membrane Assay (BBM)

Kidney membrane preparation, 5 μL was added to 625 μL HEPES buffer (25 mM) in a 1.5 mL polypropylene microcentrifuge tube with an O-ring seal to prevent evaporation. Peptide stock solution, 70 μL (300 μM in 50% acetonitrile) was carefully added to this and mixed by brief manual shaking. Next, the solution was aliquoted into six tubes (6×100 μl). One tube contained 200 μL Stop solution (50% acetonitrile and 1% formic acid) that was used to determine the initial concentration of peptide (t=0). All six aliquots were then incubated at 37° C. while mixing at 500 RPM in the Vortemp 56 incubator. The digestion was stopped at each desired time-point by adding 200 μL stop solution at one hour intervals for brush border membranes. The tube was then placed back in to the incubator until the last time-point was stopped typically 5 hours for brush border membranes. The solution was centrifuged at 19000×g for 10 minutes at room temperature. The supernatant was removed and 200 μL supernatant was transferred to an autosampler vials for analysis.

Sample analysis was done on a Sciex API 150 EX single quadrupole mass spectrometer using gradient 5-95% acetonitile in water containing 0.1% trifluoroacetic acid over 3 min. The most intense ion of the intact peptide was monitored to determine the rates of degradation and to quantify the amount of intact peptide remaining in the solution. Exemplary results are given in Table 5

### Example 6

### Assessment of Stability by DPP IV Assay

DPP-IV, approximately 5 mUnit in 5 mM Tris-HCl was added to 625 μL HEPES buffer (25 mM) in a 1.5 mL polypropylene microcentrifuge tube with an O-ring seal to prevent evaporation. Peptide stock solution, 70 μL (300 μM in 50% acetonitrile) was carefully added to this and mixed by brief manual shaking. Next, the solution was aliquoted into six tubes (6×100 μl. One tube contained 200 μL Stop solution (50% acetonitrile and 1% formic acid) that was used to determine the initial concentration of peptide (t=0). All six aliquots were then incubated at 37° C. while mixing at 500 RPM in the Vortemp 56 incubator. The digestion was stopped at 10 min intervals by adding 200 μL stop solution. The tube was then placed back into the incubator for 50 min. The solution was centrifuged at 19000×g for 10 minutes at room temperature. The supernatant was removed and 200 μL supernatant was transferred to an autosampler vials for analysis.

Sample analysis was done on a Sciex API 150 EX single quadrupole mass spectrometer using gradient 5-95% acetonitile in water containing 0.1% trifluoroacetic acid over 3 min. The most intense ion of the intact peptide was monitored to determine the rates of degradation and to quantify the amount of intact peptide remaining in the solution. Exemplary results are given in Table 5.

TABLE 5

| | | | |
|---|---|---|---|
| | | BBM | DDP IV |
| SEQ | | % | % |
| ID NO | Sequence | Stable | Stable |

| | | | |
|---|---|---|---|
| 1 | HAEGTFTSDVTQQLDEKAAKEFIDWLINGGP | 98 | 99 |
| | SKEIIS-OH | | |
| 3 | HAEGTFTSDVSSYLEGQAAKEFIAWLVKG | 15 | 27 |
| | R-NH2 | | |

All publications, patents, patent applications, and other references cited in this application are herein incorporated by reference in their entirety as if each individual publication, patent, patent application, or other reference was specifically and individually indicated to be incorporated by reference.

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the scope of the following claims.

1-68. (canceled)

69. An isolated non-mammalian GLP-1 peptide comprising the amino acid sequence of any one of SEQ ID NOs. 6-47 and 53-56.

70. The peptide of claim 69, comprising the amino acid sequence of any one of SEQ ID NOs. 6-28, 31-36, 38, 40-45, and 53-56.

71. The peptide of claim 69, comprising the amino acid sequence of any one of SEQ ID NOs. 14-18, 26-28, 32, 34-36, 40, 42, 43, and 53-56.

72. The peptide of claim 69 comprising the amino acid sequence of SEQ ID NO. 31, 33, 38, 41, 44, or 45.

73. The peptide of claim 69, further comprising a polyamino acid, a fatty acid, a polyethylene glycol, albumin, gelatin, or an antibody.

74. The peptide of claim 69, further comprising a polyethylene glycol having a molecular weight of 500 to 20,000.

75. An isolated polynucleotide that encodes the peptide of claim 69.

76. A pharmaceutical composition comprising the peptide of claim 69 and a pharmaceutically acceptable carrier.

77. A method of treating a mammal having a condition for which administration of a GLP-1 peptide is indicated, wherein the method comprises administering to the mammal a therapeutically effective amount of the peptide of claim 69.

78. A method for reducing appetite, reducing food intake, treating obesity, stimulating insulin secretion, treating diabetes, treating irritable bowel syndrome, treating acute lung injury, treating respiratory distress syndrome, treating cor pulmonale, treating chronic obstructive pulmonary disease, or treating sepsis in a mammal in need thereof comprising administering to the mammal a therapeutically effective amount of the peptide of claim 69.

79. The method of claim 78, wherein the mammal is a human.

80. The method of claim 78, wherein the administration is a single intravenous injection, continuous intravenous administration, a single subcutaneous injection, continuous subcutaneous administration, a regimen of multiple subcutaneous injections, a micropressure injection system, an ambulatory pump, a depot sustained-release injection, an implant, a deep lung sustained-release insufflation, a skin patch, a buccal patch, or a sustained-release oral delivery dose form.

81. The method of claim 78, wherein the diabetes is type 1 diabetes or type 2 diabetes.

82. A method to reduce the morbidity and mortality associated with myocardial infarction, stroke, a catabolic change that occurs after surgery, or respiratory distress in a mammal in need thereof comprising administering to the mammal a therapeutically effective amount of the peptide of claim 69.

83. A pharmaceutical composition comprising (i) a peptide which comprises the amino acid sequence of any one of SEQ ID NOs. 1, 2, and 6-56; and (ii) a pharmaceutically acceptable carrier.

84. A method of treating a mammal having a condition for which administration of a GLP-1 peptide is indicated, wherein the method comprises administering to the mammal a therapeutically effective amount of the pharmaceutical composition of claim 83.

85. A method for reducing appetite, reducing food intake, treating obesity, stimulating insulin secretion, treating diabetes, treating irritable bowel syndrome, treating acute lung injury, treating respiratory distress syndrome, treating cor pulmonale, treating chronic obstructive pulmonary disease, or treating sepsis in a mammal in need thereof comprising administering to the mammal a therapeutically effective amount of the pharmaceutical composition of claim 83.

86. A method to reduce the morbidity and mortality associated with myocardial infarction, stroke, a catabolic change that occurs after surgery, or respiratory distress in a mammal in need thereof comprising administering to the mammal a therapeutically effective amount of the pharmaceutical composition of claim 83.

87. An isolated peptide having at least 90% sequence identity to a peptide comprising the amino acid sequence of any one of SEQ ID NOs. 6-47 and 53-56.

88. A method for increasing stability or resistance to degradation of a subject peptide comprising either:
(i) adding an amino acid sequence comprising 5 or more sequential amino acids from a GLP-1 peptide to the C-terminus of the subject peptide; wherein the GLP-1 peptide comprises the amino acid sequence of SEQ ID NO. 57, 58, 59, 60, 61, 62, 63, 64, 65, 66, 67, or 68; or
(ii) replacing 5 or more sequential C-terminal amino acids of the subject peptide with a corresponding number of sequential amino acids from a GLP-1 peptide; wherein the GLP-1 peptide comprises the amino acid sequence of SEQ ID NO. 57, 58, 59, 60, 61, 62, 63, 64, 65, 66, 67, or 68.

89. The method of claim 88, wherein the subject peptide is a mammalian peptide.

90. The method of claim 88, wherein the subject peptide is (i) an exendin-4 peptide or (ii) a human GLP-1 peptide.

91. A peptide produced by the method of claim 88.
